(19) United States
(12) Patent Application Publication
(10) Pub. No.: US 2025/0270161 A1
(43) Pub. Date: Aug. 28, 2025

(54) HETEROGENEOUS CATALYTIC PROCESS FOR MANUFACTURING ALKYL GALLATES

(71) Applicant: Sravathi Advance Process Technologies Private Limited, Bengaluru (IN)

(72) Inventors: Dooganavar Tara, Belagavi (IN); Ganapathy Bhotla Venkata Ramanarayanan, Bangalore (IN); Puttaramaiah Raghu, Bangalore (IN); Shetty Tirumalesh, Bhatkal (IN); Anapat Samir, Bangalore (IN)

(73) Assignee: Sravathi Advance Process Technologies Private Limited, Bengaluru (IN)

(21) Appl. No.: 19/060,732

(22) Filed: Feb. 23, 2025

(30) Foreign Application Priority Data
Feb. 23, 2024 (IN) 202341013094

(51) Int. Cl. C07C 67/08; C07C 67/48

(52) U.S. Cl. CPC C07C 67/08; C07C 67/48

(57) ABSTRACT

A heterogeneous catalytic process for manufacturing alkyl gallates/digallates (e.g., esters of gallic acid) from gallic acid (e.g., 3,4,5-trihydroxy benzoic acid) and corresponding C1-C20 alkyl alcohol/diol in the presence of a non-halogenated, hydrophilic, strong cationic ion exchange resin, with high purity and higher yields. The process is devoid of neutralization and de-colorization steps and is free of toxic solvents and is suitable to synthesize higher alkyl gallates which are otherwise difficult to obtain by conventional methods.

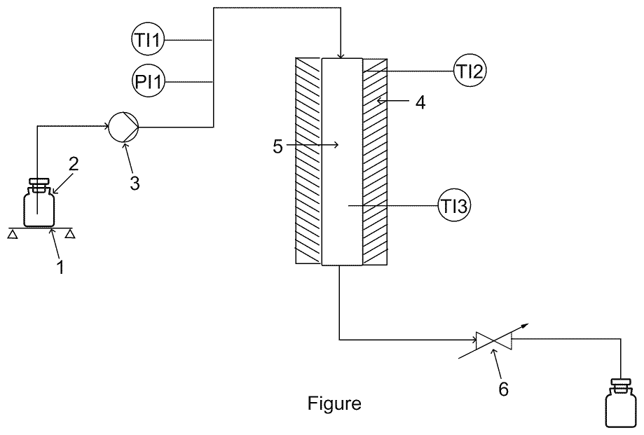

### CROSS-REFERENCE TO RELATED APPLICATION

The instant application claims priority to Indian Patent Application number 202341013094, filed Feb. 23, 2024, the entire specification of which is expressly incorporated herein by reference.

### FIELD OF THE INVENTION

The present invention relates to the manufacturing of alkyl gallates (alkanol esters of gallic acid) and more particularly relates to a process of manufacturing alkyl gallates from alkyl alcohols/diols, and gallic acid (3,4,5-trihydroxy benzoic acid) by way of employing a strong cationic exchange resin as a heterogeneous catalyst.

### BACKGROUND OF THE INVENTION

Gallic acid or 3,4,5-trihydroxy benzoic acid is a natural product which is present abundantly in many plant species. Gallic acid reacts readily with alcohols forming esters called alkyl gallates in presence of strong acid catalysts. The esterification of the gallic acid with lower alcohols, C5-C9 alkyl alcohols, diols and higher alkyl alcohols has attained significant importance as these esters find extensive application as food-preservatives, food-additives, as preservatives in pharmaceuticals and cosmetics. Alkyl gallates also find considerable application in electronics, sensors owing to their inherent ability to absorb the incident radiation. The synthetic route for producing alkyl gallates definitely needs the presence of a H source to catalyse the reaction for obtaining maximum yields. Literature search discloses many such sources that can supply protons to catalyse this reaction such as H₂SO₄, p-toluene-sulfonic acid, KHSO₄, benzene sulfonic acid and other strong proton donors.

Alkaline conditions, exposure to oxygen, ROS, metals such as iron, light, heat can severely dampen the oxidizing property of gallic acid esters, in which case the preparation of gallic acid esters or gallates necessitates a careful and clever selection of the H source in order to achieve maximum conversion, stability, and prevention of negative reactions that can reduce overall efficacy of such esters. Prior art search throws light on many reports that employed various H⁺ sources to catalyse the esterification of gallic acid with lower and higher alkyl alcohols to obtain alkyl gallates.

PCT Patent Application Publication No. WO2001030299A2 disclosed a method of synthesizing alkyl gallates from gallic acid and an alkyl alcohol of the required order of the desired gallate by heating in a reaction vessel in the presence of a catalyst such as sulfuric acid or para-toluene sulfonic acid, HCl but the yields were not appreciable.

U.S. Pat. No. 4,613,683 disclosed a method of preparation of C1-C20 alkyl gallates with 60-80% purity by reacting tannin containing materials such as hydrolysable tanning agents, which contain water-soluble esters of gallic acid, and digallic acid with sugars, with the corresponding alcohol R—OH and a strong acid which needed further recrystallization from toxic solvents.

Because gallic acid is abundantly available in the nature in many plant sources like apples, leaves, bitter almonds, and roots, many studies have been taken up to convert such plant-based gallic acid into alkyl gallates in presence of enzymes like tannases, lipases, esterases obtained from fungi like Aspergillus niger, bacteria like Bacillus licheniformis, etc.

Sharma et al. (SCD115012015-01-01PubMed) reported Gallic acid-based alkyl esters synthesis in a water-free system by celite-bound lipase of Bacillus licheniformis especially, methyl, ethyl, n-propyl and n-butyl gallates but the yields were not appreciable.

Obtaining alkyl gallates especially propyl, octyl, dodecyl and nonyl gallates of high purity, with white crystalline appearance, necessitates the use of homogenous acid catalysts. Any process that employs a homogenous acid catalyst will eventually lead to corrosion causing exposure to iron leading to a colored product. Such a process also requires neutralization of the acid catalyst downstream with a strong base exposing alkyl gallates to basic conditions, under which they are not stable.

Hence it is desirable to develop a synthetic process that employs a strongly acidic heterogenous catalyst such as an ion exchange resin (IER) that reduces the risk of corrosion and avoids the need for neutralization at the down-stream. Moreover, current methods for the synthesis of higher alkyl gallates (n>5) are rather impractical for large production purposes and/or require toxic and expensive solvents to azeotropically remove the water formed in case of such synthesis by homogeneous acid catalysis. There is, accordingly, a need for a highly selective, cost-effective, neutralization-step-free synthesis method for the production of C1-C20 alkyl gallates, particularly higher order gallates, along with lower alkyl gallates with high purity and considerable yields using strong cationic exchange resin as a heterogeneous acidic catalyst. Prior art contained very few references with regard to the use of IERs for the synthesis of alkyl gallates.

Wen et al. (Strong-acid cation exchange resin Catalytic Synthesis of Propyl Gallate [J], Speciality Petrochemicals is in progress, 2003, 4 (7): 7-9) reported utilization of sulfonate resins D72, D61 and D001-CC to catalyze and synthesize propyl gallate, but with a productive rate of only 80%, and the resin after 6 times of reuse lost its efficiency to lower than 20%.

Chinese Patent No. 105294433A disclosed a synthetic method of gallic acid lower alkanol esters comprising the steps of: (1) placing gallic acid and lower alkanol in a reactor, adding a sulfonic acid resin catalyst subjected to hydrophobic modification, carrying out stirring, heating to a temperature of 65 to 120° C., performing the reaction for 4 to 10 hours and filtering to obtain filtrate; (2) removing excessive alcohol in the filtrate by evaporation so as to obtain a crude product, then carrying out recrystallization by deionized water, and carrying out suction filtration and drying to obtain the gallic acid lower alkanol ester. The patent also included decolouration of the final product using activated carbon and is restricted to lower alkanol esters of gallic acid.

Nguyen et al. made a mention of brominated sulfonic acid resin use for obtaining propyl gallate in their Short Note on Propyl Gallate (Hoa Binh Nguyen et al in Propyl Gallate. Molbank 2021, 2021, M1201 (https://doi.org/10.3390/M1201)).

In view of the above prior art references, a cost-effective, environmentally friendly process that employs non-halogenated, hydrophilic, strong cationic exchange resin to synthesize C1-C20 alkyl gallates, that includes both lower and higher alkyl gallates is the need of the hour. Moreover, such heterogeneous catalytic process should be free from the usage of toxic solvents for recrystallization, devoid of decolourization step and be giving maximum yields of such alkyl gallates of high purity. Keeping these points in mind the present invention of “Heterogeneous catalytic process for manufacturing alkyl gallates” to synthesize C1-C20 alkyl gallates from gallic acid and corresponding alkyl alcohol/diol in presence of a non-halogenated and hydrophilic strong cationic exchange resin is taken up. The process is devoid of neutralization step and further prevents decolourization step due to the absence of alkali which is required for neutralization downstream.

The exemplary aspects of various embodiments of the present invention are disclosed in the summary of the invention and all the essential aspects related to various embodiments of the invention are described in a detailed manner in the following paragraphs with specific references towards the corresponding figures as given hereunder. All of the prior art references are incorporated herein in their entirety for reference-sake and in no way taking away the novelty of the present invention. The various aspects of the present invention disclosed here are definitely an improvement over the existing prior art and further stress upon the inventorship, novelty and applicability of the present invention.

### Objectives of the Invention

To come out with a heterogeneous catalytic process of manufacturing alkyl gallates (esters of gallic acid) from gallic acid (3,4,5-trihydroxy benzoic acid) and alkyl alcohol/diol in the presence of a non-halogenated, hydrophilic strong cation exchange resin.

To come out with a heterogeneous catalytic process of manufacturing alkyl gallates in presence of a strong cationic exchange resin which is devoid of neutralization at the down-stream, de-colorization step, and cost-effective.

To come out with a heterogeneous catalytic process for manufacturing alkyl gallates with high purity, higher yields in presence of a strong cationic exchange resin that facilitates the synthesis of C1-C20 alkyl gallates without the use of toxic solvents for crystallization.

### SUMMARY OF THE INVENTION

The exemplary embodiment of the present invention discloses a heterogeneous catalytic process for manufacturing alkyl gallates (esters of gallic acid) from gallic acid (3,4,5-trihydroxy benzoic acid) and corresponding C1-C20 alcohol/diol in the presence of non-halogenated, hydrophilic, strong cationic exchange resin (IER), that is devoid of neutralization at the downstream and is further devoid of decolourization step in a continuous mode in a packed-bed-reactor.

Another important embodiment of the present invention discloses a heterogeneous catalytic process for manufacturing alkyl gallates (esters of gallic acid) from gallic acid (3,4,5-trihydroxy benzoic acid) and corresponding C1-C20 alcohol/diol in the presence of non-halogenated, hydrophilic, strong cationic exchange resin (IER), that is devoid of neutralization at the downstream and is further devoid of decolourization step in a batch reactor.

One embodiment of the present invention discloses a heterogeneous catalytic process for manufacturing alkyl gallates (esters of gallic acid) from gallic acid (3,4,5-trihydroxy benzoic acid) and corresponding C1-C20 alcohol/diol in the presence of non-halogenated, hydrophilic, strong cationic exchange resin (IER), that is devoid of neutralization at the downstream and is further devoid of decolourization step wherein the IER is having different cross-linking densities.

Yet another important embodiment of the present invention discloses a 3-stage purification of the crude product that is isolated at the downstream of the heterogeneous catalytic manufacturing of alkyl gallates from gallic acid and corresponding C1-C20 alcohol/diol in the presence of a non-halogenated, hydrophilic IER, after the removal of the alcohol and washing with deionised water.

One important embodiment of the present invention discloses the assaying of the alkyl gallates thus obtained by the heterogeneous catalytic process of manufacturing from gallic acid and C1-C20 alcohol/diol in the presence of non-halogenated, hydrophilic IER.

The various embodiments of the present invention are described in detail in the following paragraphs.

### BRIEF DESCRIPTION OF DRAWINGS

The FIGURE illustrates a schematic view of the experimental set-up comprising the packed bed reactor for continuous manufacturing of alkyl gallates, in accordance with the general teachings of the present invention.

### DETAILED DESCRIPTION OF THE INVENTION

The examples of the apparatus discussed herein are not limited in application to the details of construction and the arrangement of components set forth in the following description or illustrated in the accompanying drawings. It will be understood by one of skill in the art that the apparatus is capable of implementation in other embodiments and of being practiced or carried out in various ways. Examples of specific embodiments are provided herein for illustrative purposes only and are not intended to be limiting. Also, the phraseology and terminology used herein is for the purpose of description and should not be regarded as limiting.

Any references to examples, embodiments, components, elements or acts of the apparatus herein referred to in the singular may also embrace embodiments including a plurality, and any references in plural to any embodiment, component, element, or act herein may also embrace embodiments including only a singularity (or unitary structure). References in the singular or plural form are not intended to limit the presently disclosed apparatus, its components, acts, or elements.

The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms. The various embodiments of present invention are described in detail herein and the various aspects of the present invention are disclosed below.

### Definitions

GA is Gallic acid.

Alkyl gallate is an ester formed by the reaction of gallic acid and an alkyl alcohol or diol.

IER is a strong cationic ion exchange resin.

PG is propyl gallate.

‘meq’ is milli equivalents of the ion exchange resin.

TULSION is the commercial name of the ion exchange resin used.

Packed bed reactor is a heterogeneous reaction system consisting of a cylindrical shell with convex heads with the interior containing an immobilized, or fixed, bed of catalyst.

WHSV is weight hourly space velocity.

HPLC is High performance liquid chromatography.

GC is gas phase chromatography.

FT-IR is Fourier transform infrared spectroscopy.

UV is ultraviolet spectroscopy.

“barg” is operating pressure in excess of one atmosphere.

Deionized water is water devoid of dissolved ions.

Heterogeneous catalysis is a catalytic process wherein reactants, catalysts are in different phases.

Catalyst is any substance that can influence the reaction rate without participating in the reaction.

SM is starting material.

A % is Area percentage.

### Strong Cationic Exchange Resin (IER) Characteristics

The IER used was a strong cationic exchange resin consisting of sulfonated polystyrene cross-linked with 2-8% of divinyl benzene with an exchange capacity of >5 meq/g (dry resin). The commercial names of the resins employed are TULSION T3825 and T-66 which differed with respect to the % of cross-linking and in turn their conversion capabilities. TULSION T3825, is sulfonated polystyrene cross-linked with 2% divinyl benzene, having an exchange capacity >5 meq/g (dry resin). IER T-66 is having 8% cross-link density and similar exchange capacity (was found to give lower conversion).

### Instrumental Analysis

The experimental samples are analysed by various instruments such as FT-IR, UV to arrive at the purity, yield of the products and also to estimate impurities, side-reactions if any. The conditions at which the analysis is done using different instruments are given in the following paragraphs.

### FT-IR Analysis

Done on FT-IR instrument with KBr wafers and the spectra of the samples obtained are compared with the standard reference spectra.

### UV Analysis

0.100 g of the experimental sample is dissolved in methanol and diluted to 250.0 ml with the same solvent. 5.0 ml of this diluted solution is taken in a cuvette and the absorbance is measured at the absorption maximum of 275 nm. The content of sample is calculated taking the specific absorbance as 503.

### HPLC Analysis:

HPLC analysis is done by maintaining the UV detector at 273 nm; using the column: Eclipse XDB C18 5 μm 250*4.6 mm P. No: 990967-902; mobile phase at a flow rate of 0.9 mL/min; at temperatures between 35° C. and 25° C.; using a sample volume of 5 μL; and run time as 28 minutes. Mobile phase A consisted of 0.1% Trifluoracetic acid in water; and Mobile phase B was Methanol. Methanol was used as a diluent. The retention times of Gallic acid and Propyl gallate were found to be 3.98 and 9.86, respectively.

### Method of Synthesizing Alkyl Gallates

The method of synthesizing various alkyl gallates is according to the general procedure given in the following paragraphs.

Method of synthesizing alkyl gallates was done in both continuous and batch modes by the reaction of specified amounts of gallic acid the corresponding alkyl alcohol or alkyl diol in presence of measured quantity of the strong cationic exchange resin having prescribed cross-linking and exchange capacity and, in each case, the final product was isolated and purified. The final product purity and yield were estimated by subjecting the samples to HPLC, UV and IR analyses.

### Continuous Mode of Synthesis of Alkyl Gallates Using IER as the Heterogeneous Catalyst Description of the Schematic of the Packed-Catalyst-Bed Reactor for Continuous Mode of Synthesis of Alkyl Gallates

The continuous mode of synthesis of alkyl gallates comprised of a packed bed reactor as given in the FIGURE. The schematic comprises a packed catalyst bed reactor (5) that is fed by the feed from the feed bottle (2) comprising the measured amounts of the gallic acid and the alkyl alcohol/diol corresponding to the alkyl gallate or digallate to be produced, through the feed lines. The feed heated by a hot-plate-stirrer (1) will be pumped to the packed bed reactor through a metering pump (3). The feed lines are provided with feed temperature indicator (T11), and feed pressure indicator (P11). The packed catalyst bed is provided with a casing to provide electric heating coupled with a temperature controller (4). The packed bed reactor is also provided with Reactor Electric Heat Tracing Temperature Indicator (T12), Catalyst bed temperature indicator (T13) and backpressure regulator (6).

### Continuous Mode of Synthesis of Alkyl Gallates in the Packed-Catalyst-Bed Reactor

The packed bed reactor is filled with 400 g of dry TULSION exchange resin having 2% cross-linking and an exchange capacity of 5 meq/g dry catalyst. A solution comprising measured amounts of gallic acid and alcohol corresponding to the gallate to be produced are pumped in to the reactor at 0.5 weight hourly space velocity at 65° C. The resin bed temperature was maintained at 85° C. by means of electrical heating through a heating tape wound outside the reactor. A slight backpressure (1 bar (g)) was maintained to avoid flashing of the alcohol in the reactor & feed lines. All feed/product lines are heat traced and temperature was maintained >65° C. to avoid precipitation of GA. The experiments are repeated with various amounts of gallic acid, C1-C20 alkyl alcohols and alkyl diols corresponding to the gallates to be synthesized. All the samples after the continuous mode were subjected to HPLC analysis to arrive at the purity and yield of the final product. The experiments are conducted to obtain various industrially important gallates such as methyl gallate, ethyl gallate, propyl, butyl, nonyl, octyl, dodecyl gallates, by pumping measured amounts of gallic acid and corresponding alcohols through the IER packed bed reactor and the results are tabulated. In each case assaying of the alkyl gallates was done to estimate the purity standards of the final product according to EP specifications, effect of no. of crystallizations on purity of the final product, amount of water used for recrystallization on the purity of the final product were measured and the results are tabulated.

### Batch Mode of Synthesis of Alkyl Gallates Using IER as the Heterogeneous Catalyst

Comparative efficiency of the IER as the heterogeneous catalyst for synthesizing alkyl gallates was also established by conducting the alkyl gallate synthesis in batch mode. Measured amounts of gallic acid and alcohol corresponding to the alkyl gallate to be synthesized along with specified amount of dry IER were heated to 100-105° C. and held at that temperature in a sealed tube with stirring for 5-20 hours. At the end of the experiment, the mass was cooled and IER was filtered out and the mass was analysed by HPLC. Excess alcohol was evaporated in a rotary evaporator to obtain a crude batch product. This crude batch product was analysed by HPLC for estimating the purity of final product.

### Procedure for Isolation & Purification of the Alkyl Gallates Synthesized Using IER as the Heterogeneous Catalyst

1000 g of reactor effluent was collected for downstream isolation and purification. The alkyl gallate was isolated from the reactor effluent by evaporating the alcohol under vacuum at a temperature of 55-60° C. To ensure complete removal of alcohol, 408 mL of deionized water was added, and the remaining alcohol was removed along with water as an azeotrope.

544 mL of deionized water was added to the crude product and the mass was heated to 65-75° C. and stirred until a clear solution was obtained. The solution was cooled gradually. Solids were seen precipitating on reaching a temperature of 40° C. The mass was cooled to 33-36° C. and stirred for 30-40 minutes. The slurry thus obtained was filtered under vacuum and the cake was washed with 136 mL deionized water. Vacuum was applied until all the free water was drained from the cake (first purification product).

To the first purification product, 544 mL deionized water was added, and the mixture was heated to 74-76° C., stirred for 30-40 minutes to obtain a clear solution. This solution was filtered hot through a WHATMAN 42 filter paper. Filtrate was slowly cooled under stirring to 32-35° C., on reaching 32-35° C. temperature was maintained and stirring is continued for 60 minutes. The slurry thus obtained was filtered under vacuum and the cake was washed with 136 mL deionized water. Vacuum was applied until all the free water was drained from the cake (the second purification product). The second purification product was dried in a vacuum oven for 7-8 hours at 55-58° C. with intermittent crushing of lumps. Samples were analyzed every 3 hours for loss of weight on drying (LOD), until LOD is <0.5%.

### Assaying Methods (EU-Specifications)

All the alkyl gallates obtained at the end of final purification were subjected to assaying to conform to the EU specifications according to the following methods and the results are tabulated.

Estimation of gallic acid: Examined by thin-layer chromatography.

Total chlorine was estimated according to the following procedure. 0.5 g of the alkyl gallate was mixed with 2 g of calcium carbonate. Dried and ignited at 700±50° C. The residue was taken up with 20 ml of dilute nitric acid and diluted to 30 ml with water. 15 ml of the solution, without further addition of dilute nitric acid, was subjected to chloride test to comply with the limit for chlorides (200 ppm).

Chlorides were estimated according to the following procedure. 50 ml of water was added to 1.65 g of the alkyl gallate, shaken for 5 minutes, and filtered. 15 ml of the filtrate was subjected to chloride test to comply with the limit for chlorides (100 ppm).

Zinc was determined by atomic absorption spectrometry to comply with the limit of not more than 25.0 ppm of Zn.

Heavy metals were estimated taking 2.0 g of final product in respect of the reference solution containing 2 ml of lead standard solution (10 ppm Pb) to comply with the limit for heavy metals (10 ppm).

### Procedure for Measuring Loss on Drying

A wide mouth weighing bottle along with lid was dried at 105° C. for 30 minutes. The weighing bottle was cooled in a desiccator for 30 minutes and the weight of the weighing bottle along with lid was recorded (W1 g). About 1 g of the sample was transferred into the weighing bottle, closed with the lid and the weight was recorded (W2 g). The material was spread to a thickness of 5 to 10 mm by shaking the bottle sideways. The weighing bottle along with the sample was kept in the oven at 105° C. and the lid was kept separately. The test sample was dried in the oven for 4 hours. The weighing bottle was taken out from the oven after closing with the lid and cooled in a desiccator for 30 minutes, weighed and the weight was noted down (W3 g).

The process of drying, cooling, and weighing should be continued till the two consecutive weights, do not differ by more than 0.5 mg. Note down the final weight and calculate the percentage of loss on drying. Loss on drying of the alkyl gallate obtained was given by the equation:

$$a . ( {W}_{2 -} {W}_{3} )$$

$$Loss on drying ( \% , w / w ) = -- -- -- -- -- -- -- -- \times 100$$

$$b . ( {W}_{2 -} {W}_{1} )$$

and the results are tabulated.

Melting point determination was done after drying the sample at 110° C. for four hours and was found to be between 146° C. and 150° C.

The heterogeneous catalytic synthesis of various alkyl gallates is carried out using the IER in both continuous and batch modes and in each case the results are tabulated. The experiments are repeated to get C1-C20 alkyl gallates using corresponding alkyl alcohols and diols and particularly alkyl gallates such as methyl, ethyl, propyl, butyl, octyl, nonyl, dodecyl gallates that conform to EU specifications and the results are given in the following paragraphs. The following paragraphs disclose in detail the experimentation done to arrive at the different alkyl gallates that find extensive application in food industry, electronic industry, cosmetic industry and find application in various industrial environments.

### Procedure for Synthesizing PG:

### Procedure for Continuous Reaction:

A packed bed reactor is filled with 400 g dry TULSION T3825 ion exchange resin, characterized by 2% cross-linking and an exchange capacity of 5 meq/g dry catalyst. Reactor is constructed using a 2″ diameter Schedule 80 pipe made of SS304.

A solution of 1000 g GA and 8000 mL (6400 g) n-propanol is made at 65° C. This is pumped into the reactor at a flow of 200 g/h, which translates to a WHSV (weight hourly space velocity) of 0.5. WHSV is defined feed flow (g/h)/mass of dry catalyst (g). The resin bed temperature is maintained at 85° C. by means of electrical heating through a heating tape wound outside the reactor. A slight backpressure (1 bar (g)) is maintained to avoid flashing of propanol in the reactor & feed lines. All feed/product lines are heat traced and temperature is maintained >65° C. to avoid precipitation of GA. Under the conditions described above, the reactor effluent when analysed by HPLC shows 95-97 area % propyl gallate, the rest being predominantly unconverted GA.

### Procedure for Batch Reaction:

6.35 g of n-propanol was added to 3 g gallic acid along with 3.74 g dry IER (Both TULSION T3825 and T66 were employed for batch experimentation). The mixture was heated to 100° C. and held at that temperature in a sealed tube with stirring for 10 hours. At the end of the experiment, the mass was cooled and IER was filtered out and the mass was analysed by HPLC. Excess propanol was evaporated in a rotary evaporator to obtain a crude batch product. This crude batch product was analysed by HPLC for purity.

### Procedure for Isolation & Purification:

1000 g of reactor effluent (corresponding to 136 g gallic acid in reactor feed) is collected for downstream isolation and purification.PG is isolated from the reactor effluent by evaporating n-propanol under vacuum at a temperature of 55-60° C. To ensure complete removal of n-propanol, 408 mL of deionized water is added, and the remaining propanol is removed along with water as an azeotrope. PG forms a slurry in water (crude product).

544 mL of deionized water is added to the crude product and the mass is heated to 65-75° C. and stirred until a clear solution is obtained. The solution is cooled gradually. Solids are seen precipitating once a temperature of 40° C. is reached. The mass is cooled to 33-36° C. and stirred for 30-40 minutes. The slurry thus obtained is filtered under vacuum and the cake is washed with 136 mL deionized water. Vacuum is applied until all the free water is drained from the cake (first purification product).

To the first purification product, 544 mL deionized water is added, and the mixture is heated to 74-76° C., stirred for 30-40 minutes to obtain a clear solution. This solution is filtered hot through a WHATMAN 42 filter paper. Filtrate is slowly cooled under stirring to 32-35° C., on reaching 32-35° C. temperature is maintained and stirring is continued for 60 minutes. The slurry thus obtained is filtered under vacuum and the cake is washed with 136 mL deionized water. Vacuum is applied until all the free water is drained from the cake (second purification product).

The second purification product is dried in a vacuum oven for 7-8 hours at 55-58° C. with intermittent crushing of lumps. Samples are analyzed every 3 hours for loss of weight on drying (LOD), until LOD is <0.5%. The experiments are repeated with different amounts of n-propanol, IER, IER with different cross-linking abilities and the results are given in the following tables. Assaying of the samples was done to conform to the EU specifications and the results are given in Table 3.

Experiments are conducted to estimate the effect of no of crystallizations and the amount of water used for crystallization and the results are tabulated in the following tables.

### Estimation of Purity by UV

Propyl gallate and gallic acid are the only UV active components present in the crude product, and hence the product obtained after the third purification process is subjected to UV analysis as given in the instrumental analysis and the purity is reported on an n-propanol and water free basis.

Table 1 shows >97% PG HPLC purity obtained under enumerated reaction conditions. 1000 g reactor effluent from each experiment was collected for downstream isolation and purification.

Table 2 shows that 2 water crystallizations as described above achieve >99.9% product purity. Table 3 shows that the product, thus obtained, meets all of the EP specifications. Table 4 shows the results obtained in batch reaction experiments, using two resins. The IER with higher cross-linking yields lower conversion.

Table 5 compares the product purity obtained with single and double crystallization. It is evident that double crystallization is required for achieving the EP purity specification. Table 6 shows that to obtain high purity with single crystallization requires a large amount of water, which leads to a low yield since more PG is lost with the mother liquor.

TABLE 1
Continuous Reaction Performance

| | | | | Bed | |
|---|---|---|---|---|---|
| Exp | Flow | Feed | WHSV | Temperature | PG HPLC |
| No. | (g/h) | Composition | (h⁻¹) | (° C.) | purity |
| 1 | 140 | 13.6 wt % gallic | 0.35 | 85 | 97.7% |
| | | acid, 86.4 wt % | | | |
| | | n-propanol | | | |
| 2 | 140 | 13.6 wt % gallic | 0.35 | 85 | 97.3% |
| | | acid, 86.4 wt % | | | |
| | | n-propanol | | | |
| III | 140 | 13.6 wt % gallic | 0.35 | 85 | 97.3% |
| | | acid, 86.4 wt % | | | |
| | | n-propanol | | | |

TABLE 2
Yield and purity after purification and isolation

| Exp No. | Input GA (g) | Output PG (g) | Yield (w/w) | HPLC purity |
|---|---|---|---|---|
| I | 136 | 149 | 1.09 | 99.94% |
| II | 136 | 148 | 1.08 | 99.96% |
| III | 136 | 149 | 1.09 | 99.95% |

TABLE 3
Quality of product after isolation & purification, compared against EP specifications.

| | Specification | I | II | III |
|---|---|---|---|---|
| Appearance | White to off white powder | Off-white | Off-white | Off-white |
| | | powder | powder | powder |
| Solubility | Very slightly soluble in water | Complies | Complies | Complies |

Identification

| IR | To comply with the standard | Complies | | Complies | | Complies | |
|---|---|---|---|---|---|---|---|
| | IR spectrum. | | | | | | |
| UV | To comply with standard UV | Complies | | Complies | | Complies | |
| | spectrum | | | | | | |
| HPLC | The retention time of the major | Complies | | Complies | | Complies | |
| | peak of sample preparation | | | | | | |
| | should matches with that of | | | | | | |
| | standard preparation, as | | | | | | |
| | obtained in assay. | | | | | | |
| Melting point | 146° C.-150° C. | 149.0° | C. | 149.3° | C. | 149.3° | C. |
| Loss on drying at 105° C. | Not more than 0.5% | 0.19% | | 0.32% | | 0.25% | |
| Appearance of solution | Should be clear, not more | Complies | | Complies | | Complies | |
| (5% solution in ethanol) | intensely coloured than BY5 | | | | | | |
| Total chlorine | Not more than 200 ppm | Complies | | Complies | | Complies | |
| Chlorides | Not more than 100 ppm | Complies | | Complies | | Complies | |
| Heavy metals | Not more than 10 ppm | Complies | | Complies | | Complies | |
| Zinc | Not more than 25 ppm | Complies | | Complies | | Complies | |
| Residue on Ignition | Not more than 0.1% | 0.088% | w/w | 0.021% | w/w | 0.08% | w/w |

| Assay by HPLC (On dried | Between 98 to 102% | 99.6% | 99.8% | 99.8% |
|---|---|---|---|---|
| basis) | | | | |

Related substances by HPLC

| Unspecified impurities | Not more than 0.1% | 0.015% | | 0.011% | 0.027% | |
|---|---|---|---|---|---|---|
| Total impurities | Not more than 0.15% | 0.054% | | 0.036% | 0.05% | |
| n-Propanol content by GC | Not more than 5000 ppm | 12 | ppm | ND | 10 | ppm |

TABLE 4
Batch reaction experiments using different IERs

| | | Reaction | |
|---|---|---|---|
| Exp # | IER | mass purity | Crude purity |
| IV | TULSION T3825(2% cross-linking) | PG: 95.9% | PG: 95.9% |
| | | GA: 3.8% | GA: 3.8% |
| V | TULSION T66(8% cross-linking) | PG: 87.7% | PG: 87.5% |
| | | GA: 12.1% | GA: 12.4% |

TABLE 5
Effect of number of crystallizations on product quality

| Exp | | Input | Output | HPLC |
|---|---|---|---|---|
| # | Purification method | (g) | (g) | purity |
| VI | One-time water crystallization | 20 | 17 | 99.5% |
| VII | Slurry filtration followed by | 13.6 | 12.7 | 99.7% |
| | water recrystallization | | | |
| VIII | Two-time water crystallization | 13.6 | 13.0 | 99.9% |
| IX | Two-time water crystallization | 136 | 149 | 99.95% |

TABLE 6
Effect of amount of water used in crystallization

| | | | | | | HPLC |
|---|---|---|---|---|---|---|
| Exp # | Volume of water | Input (g) | | Output (g) | | purity |
| X | 8 vol one time crystallization. | 13.6 | g | 8.4 | g | 99.7% |
| XI | 6 vol one time crystallization | 27.2 | g | 24.1 | g | 99.5% |
| XII | 4 vol one time crystallization | 136 | g | 156 | | 99.67% |
| XIII | 4 vol two- time crystallization | 136 | g | 149 | g | 99.95% |

Those skilled in the art will appreciate that the conception, upon which this disclosure is based, may readily be utilized as a basis for designing other products without departing from the spirit and scope of the present invention as defined by the appended claims. Therefore, the claims are not to be limited to the specific examples depicted herein. For example, the features of one example disclosed above can be used with the features of another example.

Furthermore, various modifications and rearrangements of the parts may be made without departing from the spirit and scope of the underlying inventive concept. For example, the geometric configurations disclosed herein may be altered depending upon the application, as may be the material selection for the components. Thus, the details of these components as set forth in the above-described examples, should not limit the scope of the claims.

The exemplary embodiments of the present invention can be realized with the help of the following examples of preparation of different alkyl gallates by way of reaction between measured amounts of gallic acid and corresponding C1-C20 alkyl alcohol or diol in presence of the strong acidic cationic exchange resin TULSION T3825 (sulfonated polystyrene cross-linked with 2% divinyl benzene, having an exchange capacity >5 meq/g (dry resin) with 2% crosslinking) as a heterogeneous catalyst. The final products are isolated, purified and subjected to assaying to conform to EU specifications as prescribed for such alkyl gallates.

### Example 1: Preparation of Propyl Gallate

6.35 g of n-propanol was added to 3 g gallic acid along with 3.74 g dry IER (TULSION T3825 with 2% crosslinking). The mixture was heated to 100° C. and held at 100° C. in a sealed tube with stirring for 10 hours. At the end of the experiment, the mass was cooled and IER was filtered out and the mass was analysed by HPLC. Excess propanol was evaporated in a rotary evaporator to obtain a crude product. This crude product was analysed by HPLC for purity and the purity was found to be 99.5%.

Overall yield of the purified propyl gallate is 1.08-1.1 g/g gallic acid.

### Example 2: Preparation of Octyl Gallate

Preparation of octyl gallate is according to the following equation:

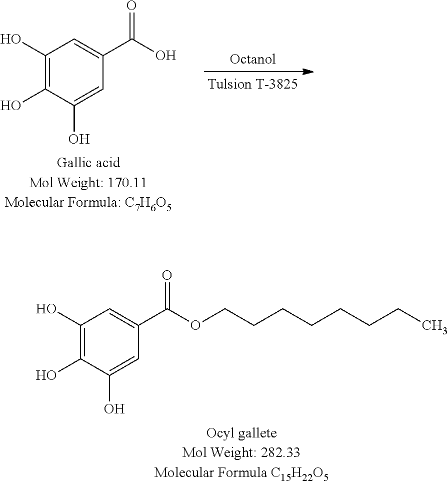 [SMILES: O=C(O)c1cc(O)c(O)c(O)c1 ; CCCCCCCCOC(=O)c1cc(O)c(O)c(O)c1]

13.78 g (6 eq) 1-octanol was added to 3 g Crude gallic acid along with 6.7 g dry IER 40% TULSION T-3825 dry (TULSION T3825 with 2% crosslinking) with removal of sample for In-Process Control check (IPC) to monitor starting material conversion and also product selectivity obtained with time at 12th hour. The mixture was heated in a sealed tube. Gallic acid and 1-octanol react with IER-T3825 in a sealed tube at temp 105° C. for 20 hours with more than 95A % purity in IPC At the end of the experiment, the mass was cooled and IER was filtered out and the mass was analysed by HPLC. Excess propanol was evaporated in a rotary evaporator to obtain a crude product. This crude product was analysed by HPLC for purity and the purity was found by IPC-HPLC; Product: 95.28A %; SM: 4.29A %; Crude purity by HPLC: Product: 97.4A %, SM: 2.3A %; Mass confirmed by LCMS.

### Example 3: Preparation of Nonyl Gallate

Preparation of nonyl gallate is according to the following equation:

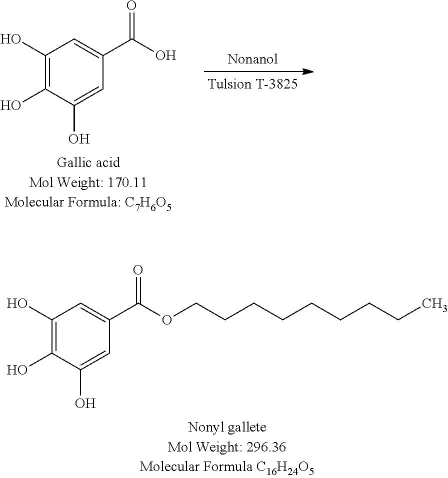 [SMILES: O=C(O)c1cc(O)c(O)c(O)c1 ; CCCCCCCCCOC(=O)c1cc(O)c(O)c(O)c1]

3.56 g (6 eq) of Nonanol was added to 0.7 g crude gallic acid along with 1.7 g dry IER 40% TULSION T-3825 dry (TULSION T3825 with 2% crosslinking) with removal of sample for IPC at 6th hour. The mixture was heated in a sealed tube. Gallic acid and Nonanol react with IER-T3825 for 9 hours heating at 100° C. under pressure in seal tube obtained more than 95% purity in IPC. At the end of the experiment, the mass was cooled and IER was filtered out and the mass was analysed by HPLC. Excess propanol was evaporated in a rotary evaporator to obtain a crude product. This crude product was analysed by HPLC for purity and the IPC purity was found by HPLC: Product: 95.22A %; SM: 4.12A %; Crude purity by HPLC: Product: 95.88 A %; SM: 3.5A %; Mass confirmed by LCMS. Gallic acid and 1-nonanol reacted with IER-T3825 in a seal tube at temp 100° C. for 9 hours obtained more than 94A % purity in IPC and crude.

### Example 4: Preparation of Dodecyl Gallate

Preparation of dodecyl gallate is according to the following equation,

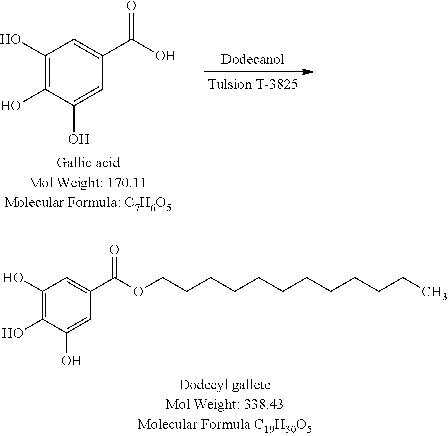 [SMILES: O=C(O)c1cc(O)c(O)c(O)c1 ; CCCCCCCCCCCCOC(=O)c1cc(O)c(O)c(O)c1]

19.7 g, (6 eq). Dodecanol was added to 3 g crude gallic acid along with 9.08 g (dry) IER 40% TULSION T-3825 dry (TULSION T3825 with 2% crosslinking) with removal of sample for IPC at 12th hour. The mixture was heated in a sealed tube. Gallic acid and 1-octanol react with IER-T3825 in a sealed tube at temp 105° C. for 20 hours obtained more than 96A % purity in IPC. At the end of the experiment, the mass was cooled and IER was filtered out and the mass was analysed by HPLC. Excess propanol was evaporated in a rotary evaporator to obtain a crude product. This crude product was analysed by HPLC for purity and the purity was found to be 20th hour IPC by HPLC: Product: 89.0A %; SM: 10.0A %; Crude purity by HPLC: Product: 89.2A %; SM: 9.9A %; Mass is confirmed by LCMS. Gallic acid and 1-dodecanol reacted with IER-T3825 in a seal tube at temp 105° C. for 20 hours to obtain more then 88A % purity in IPC and Crude.

Octyl gallate and dodecyl gallate product formation was confirmed by standard HPLC and mass by LCMS. Nonyl gallate product formation was confirmed by mass by LCMS.

The products octyl gallate, dodecyl gallate and nonyl gallate are subjected to HPLC, LCMS analyses and individual boiling points, melting points are given in Table 7 along with those of gallic acid employed in the preparation of gallates.

TABLE 7
Boiling and melting points of octyl, nonyl
and dodecyl gallates and Gallic Acid

| | | |
|---|---|---|
| Name | Boiling point | Melting point |

| | | | | |
|---|---|---|---|---|
| Gallic acid | 259.73° | C. | 251° | C. |
| Octyl gallate | 482.9° | C. | 96-102° | C. |
| Dodecyl gallate | 521.7° | C. | 94-97° | C. |
| Nonyl gallate | 492.2° | C. | 96-97° | C. |

### Advantages of the Invention

The present invention has the following advantages:

The present invention provides a heterogeneous catalytic process of manufacturing alkyl gallates in presence of a strong cationic exchange resin which is devoid of neutralization at the down-stream, de-colorization step, and cost-effective.

The present invention provides a heterogeneous catalytic process for manufacturing alkyl gallates with high purity, higher yields in presence of a strong cationic exchange resin that facilitates the synthesis of C1-C20 alkyl gallates/digallates without the use of toxic solvents for crystallization.

The present invention has excellent industrial applicability since it provides a facile method for producing higher alkyl gallates such as octyl gallate, dodecyl gallate and nonyl gallate which are otherwise difficult to prepare using conventional methods and do need considerable efforts to isolate them in pure condition and are associated with risks in conducting the reaction at high temperatures.

The present invention provides methods of obtaining C1-C20 alkyl gallates/digallates in both batch and continuous modes making use of the excellent catalytic properties of the non-halogenated, hydrophilic, strong cationic exchange resin TULSION T3825 (sulfonated polystyrene cross-linked with 2% divinyl benzene, having an exchange capacity >5 meq/g (dry resin) with high purity, yield percentage.

### Analysis of Novelty, Inventiveness and Industrial Applicability

### Novelty

The present invention is novel in the light of the prior art since it provides an excellent method for producing alkyl gallates/digallates wherein a non-halogenated, hydrophilic strong cation exchange resin of sulfonated polystyrene cross-linked with 2% divinyl benzene is employed as a catalyst to catalyse the esterification of gallic acid and C1-C20 alcohols/diols in both batch and continuous modes.

### Inventiveness/Inventive-Step/Non-Obviousness

The present invention has inventiveness and does not appear obvious for person skilled in the art for the following reasons:

The present invention provides a heterogeneous catalytic process of manufacturing alkyl gallates/digallates with high purity, higher yields in presence of a strong cationic exchange resin which is devoid of neutralization at the down-stream, decolourization-step, use of toxic solvents for crystallization and cost-effective;

The present invention provides a facile method for producing higher alkyl gallates such as octyl gallate, dodecyl gallate and nonyl gallate which are otherwise difficult to prepare using conventional methods and do need considerable efforts to isolate them in pure condition and are associated with risks in conducting the reaction at high temperatures; and

The present invention provides methods of obtaining C1-C20 alkyl gallates/digallates in both batch and continuous modes making use of the excellent catalytic properties of the non-halogenated, hydrophilic, strong cationic exchange resin TULSION T3825 (sulfonated polystyrene cross-linked with 2% divinyl benzene, having an exchange capacity >5 meq/g (dry resin) with high purity, yield percentage.

### INDUSTRIAL APPLICABILITY

The present invention has excellent industrial applicability since it provides a facile method for producing C1-C20 alkyl gallates/digallates from gallic acid and corresponding C1-C20 alcohols/diols and especially higher alkyl gallates such as octyl gallate, dodecyl gallate and nonyl gallate which are otherwise difficult to prepare using conventional methods and do need considerable efforts to isolate them in pure condition, and are associated with risks in conducting the reaction at high temperatures.

What is claimed is:

1. A heterogeneous catalytic process for manufacturing C1-C20 alkyl gallates/digallates from gallic acid and a corresponding C1-C20 alkyl alcohol/diol in the presence of a non-halogenated, hydrophilic, strong cationic exchange resin in a continuous packed-bed reactor or a batch reactor, comprising the steps of:
(a) loading of a composition containing measured amounts of gallic acid, C1-C20 alkyl alcohol/diol corresponding to an alkyl gallate/digallate to be synthesized, and a dry cationic exchange resin;
(b) heating of the loaded composition at temperatures greater than 65° C. in the case of the continuous packed-bed reactor or heating of the loaded composition at temperatures of 100-105° C. for a period of 5-25 hours in the case of the batch reactor;
(c) isolation of the alkyl gallate/digallate containing crude product produced from a reactor effluent;
(d) cooling of the crude product containing alkyl gallate/digallate; and
(e) purification of the isolated crude alkyl gallate/digallate.

2. The heterogeneous catalytic process as claimed in claim 1, wherein the dry cationic exchange resin is sulfonated polystyrene cross-linked with 2-8% of divinyl benzene with an exchange capacity of greater than 5 meq/g.

3. The heterogeneous catalytic process as claimed in claim 1, wherein the isolation of the crude alkyl gallate/digallate from the reactor effluent is by way of evaporating any remaining alcohol and washing with deionised water.

4. The heterogeneous catalytic process as claimed in claim 1, wherein the crude product containing alkyl gallate/digallate from step (c) is subjected to cooling between 30° C. and 36° C.

5. The heterogeneous catalytic process as claimed in claim 1, wherein step (e) comprises three stages of purification of the product obtained from the cooling step, wherein:
(a) stage one comprises washing the product from the cooling step with deionized water, followed by vacuum application until any free water is drained to obtain a first purification product;
(b) stage two comprises addition of deionized water to the first purification product, heating to 74-76° C., with stirring for 30-40 minutes to obtain a clear solution, filtering the solution through a WHATMAN 42 filter paper to obtain a filtrate, cooling the filtrate slowly under stirring to 30-35° C. for 1-2 hours to obtain a slurry, filtering the slurry under vacuum to obtain a product cake, washing the product cake with deionized water, followed by vacuum application until any free water is drained from the product cake to obtain a second purification product; and
(c) stage three comprises vacuum drying of the second purification product in an oven for 7-8 hours at 55-58° C. with intermittent crushing of any lumps.

6. The heterogeneous catalytic process as claimed in claim 1, wherein propyl gallate is produced when propyl alcohol and gallic acid are used.

7. The heterogeneous catalytic process as claimed in claim 1, wherein octyl gallate is produced when octyl alcohol and gallic acid are used.

8. The heterogeneous catalytic process as claimed in claim 1, wherein nonyl gallate is produced when nonyl alcohol and gallic acid are used.

9. The heterogeneous catalytic process as claimed in claim 1, wherein dodecyl gallate is produced when dodecyl alcohol and gallic acid are used.
